(19) United States
(12) Patent Application Publication
(10) Pub. No.: US 2014/0335615 A1
(43) Pub. Date: Nov. 13, 2014

(54) METHODS OF USING MECHANICAL FORCE WITH SOMATIC AND PLURIPOTENT CELLS

(71) Applicant: LIFE TECHNOLOGIES CORPORATION, Carlsbad, CA (US)

(72) Inventor: Pauline Lieu, San Diego, CA (US)

(21) Appl. No.: 14/370,726

(22) PCT Filed: Jan. 4, 2013
(86) PCT No.: PCT/US13/20372

§ 371 Date: Jul. 3, 2014

Related U.S. Application Data
(60) Provisional application No. 61/583,553, filed on Jan. 5, 2012.

(51) Int. Cl. C12N 5/074

(52) U.S. Cl. CPC C12N 5/0696
USPC 435/377; 435/404

(57) ABSTRACT

Provided are methods useful in increasing efficiency of biotransport, reprogramming or altering a cell's state of differentiation, and maintenance of cells in an undifferentiated state.

### CROSS REFERENCE TO RELATED APPLICATIONS

This application is a U.S. National Application filed under 35 U.S.C. §371 of International Application No. PCT/US2013/020372 filed Jan. 4, 2013, which claims priority benefit under 35 U.S.C. § 119(e) to U.S. Provisional Patent Application No. 61/583,553, filed Jan. 5, 2012, the disclosures of which are hereby incorporated by reference in their entirety as if set forth fully herein.

### TECHNICAL FIELD

The present invention relates to methods of using mechanical force(s) for biotransport, reprogramming or altering a cell's state of differentiation, and maintenance of cells in an undifferentiated state.

### BACKGROUND

The generation of induced pluripotent stem cells (iPSCs) from fibroblasts or other somatic cells enables the possibility of providing disease-specific and patient-specific iPSCs for drug screening, disease modeling, and cell therapy applications. For example, Takahashi et al. demonstrate reprogramming of differentiated human somatic cells into a pluripotent state through the introduction of four factors, Oct3/4, Sox2, Klf4, and c-Myc (Cell, 131:1-12, 2007). The use of iPSCs is made somewhat difficult by the low efficiency of iPSC derivation, ranging, for example, from 0.0001% to 1% efficiency depending on different delivery methods and cell types. Further limiting the generation and application of patient-specific iPSCs is the observation that adult somatic cells are more difficult to reprogram, with significantly lower efficiency, than neonatal or fetal cells.

### SUMMARY

Provided herein, in part, are improved methods for preparing an induced pluripotent stem cell (iPSC) by reprogramming a somatic cell. Also provided are methods for increasing the efficiency of somatic cell reprogramming to iPSCs. Also provided herein are cell compositions derived from somatic cells in which at least 1% of the cells in the population are iPSCs.

Also provided are methods for increasing efficiency of inducing differentiation of a pluripotent cell. Also provided are methods for increasing efficiency of inducing transdifferentiation of a somatic cell. Also provided are methods for increasing efficiency of inducing transdifferentiation of a somatic cell. Also provided are improved methods for maintenance of cells in an undifferentiated state. Also provided are methods for increasing the efficiency of biotransport in cells.

In one aspect, provided herein is a method for preparing an iPSC by reprogramming a somatic cell, the method comprising imposing mechanical force on a somatic cell in culture and contacting the cell with at least one reprogramming factor. In some embodiments of the method, the resultant cell population comprises greater than 1% of the cells being iPSCs.

In another aspect, provided herein is a method for increasing efficiency of inducing an iPSC from a somatic cell, the method comprising imposing mechanical force on a somatic cell in culture and contacting the cell with at least one reprogramming factor, so that the number of iPSCs produced is greater than in the absence of the mechanical force.

In another aspect, provided herein is a method for increasing efficiency of inducing differentiation of an iPSC, the method comprising imposing mechanical force on an iPSC in culture and differentiating the iPSC with at least one differentiation factor, so that the number of differentiated cells produced is greater than in the absence of the mechanical force.

In another aspect, provided herein is a method for increasing efficiency of inducing transdifferentiation of a somatic cell, the method comprising imposing mechanical force on a somatic cell in culture and transdifferentiating the cell with at least one transdifferentiation factor, so that the number of transdifferentiated cells produced is greater than in the absence of the mechanical force.

In another aspect, provided herein is a method for increasing efficiency of nucleic acid uptake by a cell, the method comprising imposing mechanical force on a cell in culture and contacting the cell with a nucleic acid molecule, so that the number of cells containing the nucleic acid is greater than in the absence of the mechanical force.

In another aspect, provided herein is a method for maintaining pluripotent cells in an undifferentiated state, the method comprising imposing mechanical force on the cell in culture wherein the pluripotency of the cell is maintained.

In some embodiments of the provided methods, the mechanical force comprises shear force. In some embodiments, the mechanical force comprises diffusion. In some embodiments, the mechanical force is transferred through a fluid, such as, for example, a cell culture medium, a physiological salt solution, or a combination thereof.

In some embodiments of the provided methods, the mechanical force from at least one of unidirectional laminar flow, constant oscillatory flow, and to-fro flow. In some embodiments, the unidirectional laminar flow or to-fro flow is pulsatile.

In some embodiments of the provided methods, the mechanical force is imposed on the cell prior to contacting the cell with the reprogramming agent(s), the differentiation agent(s), or the trans-differentiation agent(s).

In some embodiments of the provided methods, the mechanical force is imposed on the cell following contacting the cell with the reprogramming agent(s), the differentiation agent(s), or the trans-differentiation agent(s).

In some embodiments of the provided methods, the mechanical force is imposed on the cell prior to and following contacting the cell with the reprogramming agent(s), the differentiation agent(s), or the trans-differentiation agent(s).

In some embodiments of the provided methods, the mechanical force is imposed on the cell during contacting of the cell with the reprogramming agent(s), the differentiation agent(s), or the trans-differentiation agent(s).

In some embodiments of the provided methods, the reprogramming comprises contacting the cell with a viral vector encoding at least one reprogramming factor or with at least one reprogramming microRNA. In some embodiments, the method comprises reprogramming the cell with at least two reprogramming factors or at least two reprogramming microRNAs.

In another aspect, provided herein are cell compositions derived from somatic cells in which at least 1% of the cells in the composition are iPSCs.

In some embodiments of the provided methods and compositions, the pluripotent cell is an iPSC. In some embodiments, the somatic cell is a fibroblast or an endothelial cell.

### DETAILED DESCRIPTION

Hemodynamic shear forces have been demonstrated to regulate a variety of cell processes such as signaling pathways, proliferation, oxygen transport, nitric oxide level, gene expression, as well as osteogenesis in mesenchymal stem cells (MSCs). For example, pulsatile shear force has been shown to upregulate Krüppel-Like Factor 2 (KLF2) expression in cultured vascular endothelial cells. See, for example, Young et al. (2009) Arterioscler. Thromb. Vasc. Biol. 29:1902-1908.

Provided herein are methods using mechanical forces, such as hemodynamic shear forces, in biotransport and reprogramming of cells. The mechanical force can be a fluid shear force, diffusion, or any pressure that imposes tangential or radial stresses on the surface of the cell culture. Mechanical forces are applied, for example, as unidirectional laminar flow, pulsatile unidirectional laminar flow, constant oscillatory flow, pulsatile to-fro flow, static forces, and cyclic strain.

In some embodiments, mechanical forces are generated by producing positive flow in a fluid in contact with the cell population. In other embodiments, mechanical forces are generated by producing negative (or retrograde) flow in a fluid in contact with the cell population. In certain embodiments, mechanical forces are generated by an alternating combination of positive and negative fluid flow. In various embodiments, fluid flow for the mechanical force can occur continuously or at intervals, and can increase or decrease in magnitude over time.

In some embodiments, the mechanical force is transferred through a fluid and, for example, the fluid is a cell culture medium, a physiological salt solution, or a combination thereof

Methods and equipment for application of mechanical forces on cells are known in the art. For example, Guo et al. (Cir. Res. 100:564-571, 2007) describes monolayers of endothelial cells seeded on a glass plate are assembled into a parallel-plate flow channel. The flow system is kept at 37° C. and ventilated with 95% humidified air with 5% CO₂. A laminar flow is imposed with a shear stress of 12 dyne/cm² without oscillation. Optionally, an oscillatory flow is generated by the addition of an oscillator to create a shear stress of 1±5 dyne/cm² with a frequency of 1 Hz. In another example, Young et al. (Arterioscler. Thromb. Vasc. Biol. 29:1902-1908, 2009) describes the imposition of shear stress on human umbilical cord vein endothelial cells using a circulating flow system. In this system, a reciprocating syringe pump was connected to the circulating system to introduce a sinusoidal component (frequency=1 Hz) onto the shear stress. In some circumstances, pulsatile shear flow was applied to cells with a shear stress of 12±4 dyne/cm². Hastings et al. (Am. J. Physiol. Cell Physiol. 293:C1824-1833, 2007) describes the imposition of hemodynamic shear stress on endothelial cells and smooth muscle cells in coculture. Each cell type was plated on an opposite side of a Transwell culture dish and grown to confluence before forces were applied. Hemodynamic shear stress was applied to the endothelial cells through use of a cone and plate flow device with the cone submerged in culture media and rotated in close proximity to the surface of the cells. The rotation of the cone transduces momentum on the fluid and creates time-varying shear stresses on the well or cellular surfaces.

Additional methods and equipment for application of shear stresses on cells are known in the art and commercially available. See, for example, Frangos et al. (1985) Science 227:1477-1479; Inamdar et al. (2011) Biomicrofluidics 5:22213; US Pat. Application Publication No. 2011/0033933; US Pat. No. 7,811,782; and HemoShear, LLC.

In some embodiments, mechanical forces, such as hemodynamic forces, enhance cross membrane transport of nucleic acids, polypeptides, and/or small molecules in cells. In such methods, mechanical forces are applied to the cells before, during and/or after the molecule or compound for transport is added to the cells. In such methods, imposition of mechanical force enhances cross membrane transport of any type of nucleic acid, including without limitation, DNA, RNA (for example, mRNA, microRNA, siRNA, or antisense RNA), or any combination thereof In certain embodiments, such methods for enhancing biotransport are performed in the absence of transfection regents.

In some embodiments, mechanical forces, such as hemodynamic forces, enhance the conversion of somatic cells to iPSCs by imposing shear stress onto cultured cells. In such methods, the mechanical forces are imposed on the cells before, during and/or after contacting the somatic cells with a reprogramming composition suitable for reprogramming the somatic cells to iPSCs.

Accordingly, in some embodiments, the mechanical forces are imposed on the cells at the time a reprogramming composition is added to the cells. In other embodiments, the mechanical forces are imposed on the cells before a reprogramming composition is added to the cells. In some embodiments, the mechanical forces are imposed on the cells subsequent to the addition of a reprogramming composition to the cells. In some embodiments, the mechanical forces are imposed on the cells before a reprogramming composition is added to the cells but not simultaneous with the addition of the reprogramming composition to the cells.

Induced pluripotent stem cells (iPSCs) are stem cells which are produced from differentiated somatic cells that have been induced or changed, i.e., reprogrammed, into cells in a pluripotent state. iPSCs have the ability to differentiate into cells of all three germ or dermal layers: mesoderm, endoderm, and ectoderm.

Compositions for reprogramming somatic cells to form iPSCs, and methods for inducing such reprogramming, are generally known. See, for example, Takahashi et al. (2006) Cell 126:663-676; Takahashi et al. (2007) Cell 131:861-872; Stadtfeld et al. (2008) Science 322:945-949; Okita et al. (2008) Science 322:949-953; Huangfu et al. (2009) Nat. Biotechnol. 26:795-797; US Pat. Application Pub. Nos. 2010/0144031 and 2011/0028537; U.S. Pat. Nos. 8,058,065 and 8,048,999, all incorporated herein by reference. Such reprogramming can occur, for example, by forced expression of specific transcription factors including, but not limited to, the combination of Oct4, Sox2 and Klf4. Additional reprogramming factors include, without limitation, c-Myc, bFGF, SCF, TERT, Nanog, Lin28, SV40 large T antigen, Esrrb, and Tbx3. Transfection of somatic cells with RNA, such as microRNA and mRNA, have also been used to generate iPSCs.

It has also been shown that a single transcription factor may be used in reprogramming somatic cells to iPSCs with the addition of certain other small molecule pathway inhibitors. Such pathway inhibitors include, for example, the transforming growth factor-beta pathway inhibitors such as SB431542 (4[4-(1,3-benzodioxol-5-yl)-5-(2-pyridinyl)-1H-imidazol-2-yl]-benzamide), and A-83-01 (3 -(6-Methyl-2-pyridinyl)-N-phenyl-4-(4-quinolinyl)-1H-pyrazole-1-carbothioamide), extracellular signal-regulated kinases (ERK) and microtubule-associated protein kinase (MAPK/ERK) pathway inhibitors such as PD0325901 (N-[(2R)-2,3-dihydroxypropoxy]-3,4-difluoro-2-[(2-fluoro-4-iodo- phenyl)amino]-benzamide), GSK3 inhibitors such as CHIR99021 (6-((2-((4-((2,4-Dichlorophenyl)-5-(4-methyl-1H-imidazol-2-yl)pyrimidin-2-yl) amino)ethyl)amino)nicotinonitrile), the lysine-specific demethylase 1 Parnate (tranylcypromine), the small molecule activator of 3′-phosphoinositide-dependent kinase-1 (PDK1) PS48 [(2Z)-5-(4-Chlorophenyl)-3-phenyl-2-pentenoic acid], histone deacetylase (HDAC) inhibitors such as sodium butyrate and valproic acid, small molecules that modulate mitochondrial oxidation (e.g., 2,4-dinitrophenol), glycolytic metabolism (fructose 2,6-bisphosphate and oxalate), HIF pathway activation (N-oxaloylglycine and Quercetin). Zhu et al. (2010) Cell Stem Cell 7: 651-655, incorporated by reference herein, showed that Oct4 combined with Parnate and CHIR99021 was sufficient to reprogram adult human epidermal keratinocytes.

In some instances of iPSCs derived through the use of reprogramming factors, iPSCs can contain epigenetic signatures characteristic of the somatic cell or tissue of their origin. Such a residual epigenetic signature, such as a DNA methylation signature for example, can be associated with a propensity for differentiation of the iPSC along the cell lineages related to the donor cell rather than along cell lineages different from the donor somatic cell. See, for example, Kim et al. (2010) Nature 467:285-290.

In certain embodiments, iPSCs generated by the instant methods are distinct epigenetically from iPSCs generated in the absence of mechanical forces. Accordingly, in other embodiments, as compared to differentiation of iPSCs generated in the absence of mechanical forces, differentiation of iPSCs generated by the instant methods results in an increased number differentiated cells with lineages other than that of the original somatic donor cell.

As used herein, a reprogramming factor or a reprogramming composition refers to a molecule, compound or composition which can contribute to changing or inducing (i.e., reprogramming) a somatic cell into an iPSC. As described herein and known in the art, reprogramming factors or compositions may include specific transcription factors, small molecules, RNAs, and combinations thereof

Reprogramming factors can be used alone or in combinations in order to achieve reprogramming to an iPSC. In some embodiments, the somatic cell is contacted with at least one reprogramming factor, in conjunction with the mechanical force, in order to generate an iPSC. In other embodiments, at least two reprogramming factors are used. In still other embodiments, at least three reprogramming factors or at least four reprogramming factors are used, in conjunction with the mechanical force, to generate an iPSC. When used in combination, the reprogramming factors can be all of a single type (e.g., all transcription factors), or can be a mixed combination (e.g., a transcription factor in combination with a small molecule). The reprogramming factors can be added to the cell as a mixture or individually.

Methods for generating iPSCs include introducing and expressing reprogramming factor(s) in somatic cells through, for example, infecting or transfecting the cells with expression vector(s) encoding the reprogramming transcription factor(s). Such expression vectors include viral vectors and constructs including, but not limited to, lentivirus, retrovirus, adenovirus, Sendai virus, herpes virus, pox virus, adeno-associated virus, Sinbis virus, baculovirus, or combinations thereof. Other transfection or expression vectors that may be used include, for example, plasmid vectors, DNA constructs, mRNA, microRNA, siRNA, antisense RNA, and combinations thereof

In some embodiments, mechanical forces, such as hemodynamic forces, enhance differentiation efficiency of a pluripotent cell or multipotent cell into a differentiated cell or cell type, e.g., iPSCs or embryonic stem cells (ESCs), into endoderm, mesoderm or ectoderm. In such methods, the mechanical forces are imposed on the cells before, during and/or after addition of the differentiation agent(s) to the starting cell population. Agents for inducing differentiation vary and depend, in part, on the initial cell type and/or the desired differentiated cell type, and are known in the art. Such differentiating agents include, without limitation, growth factors, transcription factors and small molecules.

ESCs are a type of pluripotent stem cell derived from the inner cell mass of blastocysts. The most common examples are mouse and human ESCs. Techniques for isolating and culturing ESCs have been developed (e.g., Thomson et al. (1998) Science 282:1145-1147; Evans et al. (1981) Nature 292:154-156; Hoffman et al. (2005) Nat. Biotechnol. 23:699-708). Embryonic stem cells can be defined by the presence of certain transcription factors and cell surface markers. For example, mouse ESCs express transcription factor Oct4 and the cell surface protein SSEA-1, while human ESCs express transcription factor Oct4 and cell surface proteins SSEA3, SSEA4, Tra-1-60 and Tra-1-81.

In some embodiments, mechanical forces, such as hemodynamic forces, enhance trans-differentiation of one cell type into another cell type, e.g., fibroblasts into neurons, fibroblasts into cardiac cells. In such methods, the mechanical forces are imposed on the cells before, during and/or after addition of the trans-differentiation agent(s) to the starting cell population. Agents for inducing transdifferentiation vary and depend, in part, on the initial cell type and/or the desired differentiated cell type, and are known in the art. Such agents include, without limitation, transcription factors and small molecules. See, for example, Graf (2011) Cell Stem Cell 9:504-516.

In other embodiments, mechanical forces, such as hemodynamic forces, can be applied in culture to embryonic stem cells (ESCs) or iPSCs to sustain pluripotency and integrity of these cells in the undifferentiated state.

In further embodiments of the methods provided, the cells are exposed to hypoxic conditions before, during and/or after imposition of the mechanical force. In other embodiments, the cells are exposed to nitric oxide production before, during and/or after imposition of the mechanical force. In other embodiments, the cells are exposed to electrical intensity before, during and/or after imposition of the mechanical force.

Also provided are cell compositions prepared by the use of the disclosed methods.

In some embodiments, the cell population can include differentiated somatic cells. Such somatic cells include, for example, fibroblasts, keratinocytes, lymphocytes and blood cells. Identification and/or confirmation of iPSCs may be performed by any art-known method including, but not limited to, detection of enzymatic activity of alkaline phosphatase, positive expression of the cell membrane surface markers SSEA3, SSEA4, Tra-1-60, Tra-1-81, and/or the expression of the KLF4, Oct3/4, Nanog, or Sox2 transcription factors in the cell. iPSCs may also be identified and/or confirmed by genetic analysis methods including, but not limited to, Southern blot and/or quantitative real time PCR (qPCR) analysis.

In some embodiments, the cell population can include multipotent cells, pluripotent cells, totipotent cells, or any combination thereof. A multipotent cell (or multipotent progenitor cell) can give rise to cells from some but not all cell lineages. For example, a hematopoietic cell is a multipotent stem cell that can give rise to several types of blood cells, but not brain cells or other non-blood cells. MSCs are a type of multipotent stem cell that can differentiate into vascular endothelial cell, bone cells, fat cells and cartilage cells. A pluripotent cell can give rise to cells from any of the three germ or dermal layers: endoderm, mesoderm, ectoderm. A totipotent cell can give rise to cells of any type, including extra-embryonic tissues.

In some embodiments, pluripotent cell cultures are grown with a feeder cell layer. In other embodiments, cells are grown in defined conditions without the use of feeder cells. Feeder-free culture conditions are known in the art and are commercially available. In certain embodiments, the pluripotent cells are in feeder-free culture conditions before, during and/or after imposition of the mechanical force.

The term “feeder cell” refers to a culture of cells that grows in vitro and secretes at least one factor into the culture medium, and that can be used to support the growth of another cell of interest in culture. As used herein, a “feeder cell layer” can be used interchangeably with the term “feeder cell.” A feeder cell can comprise a monolayer, where the feeder cells cover the surface of the culture dish with a complete layer before growing on top of each other, or can comprise clusters of cells. In a preferred embodiment, the feeder cell comprises an adherent monolayer.

The cell media is formulated to sustain cell integrity and health during the culturing and the media used may vary depending on the cell types being cultured. Compounds, such a growth factors, reprogramming factors or agents, differentiation factors or agents, trans-differentiation factors or agents, may be part of the media formulation either initially or added into the cell culture environment during the course of the culture, including before, during and/or after imposition of the mechanical forces.

The cells are cultured in a vessel appropriate for the type of cell in use. As used herein, “vessel” indicates any container or holder wherein the methods disclosed herein can occur, including without limitation, single well containers, such as test tubes, flasks, plates, bioreactors, and multi-well containers such as microtiter plates of any configuration. In some embodiments, the cells are cultured on membrane supports, including semipermeable membrane supports such as Transwell® supports.

Also provided are cell compositions derived from somatic cells in which at least 1% of the cells in the composition are iPSCs. In some embodiments, the cell composition comprises at least 1.5% iPSCs. In some embodiments, the cell composition derived from somatic cells comprises >1%, >2%, >3%, >4%, >5%, >6%, >7%, >8%, >9%, or >10% iPSCs. In some embodiments, the cell composition comprises 1-5% iPSCs.

The following examples are provided by way of illustration and not by way of limitation.

### EXAMPLES

### Mechanical Force in Enhancing Biotransport Efficiency of Nucleic Acid Molecules.

Plated fibroblasts are subject to hemodynamic shear force conditions for 24-48 hours in culture medium prior to addition of a nucleic acid reporter agent. After 24-48 hours of the shear forces, plasmid DNA encoding a GFP or miRNA-labeled Cy3 is added to the culture medium and the cells are incubated for another 24 hours. The following day, the cells are collected from the culture plate. GFP expression in the cells or Cy3 incorporation into the cells is measured by flow cytometry. For control, a parallel culture of plated fibroblasts are incubated and treated with the nucleic acid agents under the same conditions but without shear forces.

### Mechanical Force in Enhancing Reprogramming Efficiency of Somatic Cells.

Human neonatal foreskin fibroblast cells are plated in culture media and following attachment of the cells to the culture dish, the cells are subject to hemodynamic shear force conditions through the flow of cell culture medium for 24-48 hours. After 24-48 hours of culturing with shear forces, the cells are transduced with the CytoTune™-iPS Reprogramming kit (a set of four Sendai viruses each carrying a reprogramming factor (i.e., Oct4, Sox2, Klf4, c-Myc) available from Life Technologies Corp.) through the course of an overnight incubation. After 24 hours of transduction, the medium containing the virus is replaced with fresh fibroblast medium and the cells cultured with the shear force conditions. About 7 days after transduction, the cells are harvested and plated on MEF feeder cell cultures, following the culturing guide lines in the CytoTune™-iPS Reprogramming kit. For a control, a culture of the fibroblasts are plated, incubated, and treated with the reprogramming agents under the same conditions as the test culture but without shear forces.

Fifteen days following transduction, reprogramming efficiency in each of the test and control cultures is evaluated through live staining of each culture with anti-Tra1-60 or anti-Tra1-81 antibodies (Life Technologies Corp.).

### Mechanical Force in Enhancing Differentiation Efficiency of Pluripotent Cells.

Human iPSCs are plated in culture media and following attachment of the cells to the culture dish, the cells are subject to hemodynamic shear force conditions through the flow of cell culture medium for 24-48 hours. After 24-48 hours of culturing with shear forces, the cells are transduced with viruses or small molecules/agent for 24 hours. After 24 hours of transduction, the medium containing the virus is replaced with fresh medium and the cells cultured with the shear force conditions for 15-21 days. For a control, a culture of the fibroblasts are plated, incubated, and treated with the reprogramming agents under the same conditions as the test culture but without shear forces.

Fifteen days following transduction, differentiation efficiency in each of the test and control cultures is evaluated through cell surface labeling and/or detection of cell type specific RNA expression.

Mechanical Force in Enhancing Trans-Differentiation Efficiency of Somatic Cells.

Human iPSCs are plated in culture media and following attachment of the cells to the culture dish, the cells are subject to hemodynamic shear force conditions through the flow of cell culture medium for 24-48 hours. After 24-48 hours of culturing with shear forces, the cells are transduced with viruses or small molecules/agent for 24 hours. After 24 hours of transduction, the medium containing the virus is replaced with fresh medium and the cells cultured with the shear force conditions for 15-21 days. For a control, a culture of the fibroblasts are plated, incubated, and treated with the reprogramming agents under the same conditions as the test culture but without shear forces.

Fifteen days following transduction, differentiation efficiency in each of the test and control cultures is evaluated through cell surface labeling and/or detection of cell type specific RNA expression.

All publications and patents mentioned in this specification are herein incorporated by reference to the same extent as if each individual publication or patent application was specifically and individually indicated to be incorporated by reference.

What is claimed is:

1. A method for preparing an induced pluripotent stem cell (iPSC) by reprogramming a somatic cell, comprising: imposing mechanical force to a somatic cell in culture and contacting the cell with at least one reprogramming factor.

2. A method for increasing efficiency of inducing an iPSC from a somatic cell, comprising: imposing mechanical force on a somatic cell in culture and contacting the cell with at least one reprogramming factor, so that the number of iPSCs produced is greater than in the absence of the mechanical force.

3. A method for increasing efficiency of inducing transdifferentiation of a somatic cell, comprising: imposing mechanical force on a somatic cell in culture and transdifferentiating the cell with at least one transdifferentiation factor, so that the number of transdifferentiated cells produced is greater than in the absence of the mechanical force.

4. A method for increasing efficiency of nucleic acid uptake by cells in a cell population, comprising: imposing mechanical force on a cell population in culture and contacting cells in the population with a nucleic acid molecule, so that the number of cells in the population containing the nucleic acid is greater than in the absence of the mechanical force.

5. The method of any one of claims 1-4, wherein the mechanical force is transferred through a fluid.

6. The method of any one of claims 1-5, wherein the mechanical force comprises fluid shear force.

7. The method of any one of claims 1-5, wherein the mechanical force comprises diffusion through a fluid.

8. The method of claim 7, wherein the fluid is a cell culture medium, a physiological salt solution, or combination thereof

9. The method of any one of claims 1-4, wherein the mechanical force results from at least one of unidirectional laminar flow, constant oscillatory flow, and to-fro flow.

10. The method of claim 9, wherein the unidirectional laminar flow or the to-fro flow is pulsatile.

11. The method of claim 1 or claim 2, wherein the mechanical force is imposed on the cell prior to the contacting with the at least one reprogramming factor.

12. The method of claim 11, wherein the mechanical force is further imposed on the cell following the contacting with the at least one reprogramming factor.

13. The method of claim 1 or claim 2, wherein the mechanical force is imposed on the cell following the contacting with the at least one reprogramming factor.

14. The method of claim 1 or claim 2, wherein the at least one reprogramming factor is encoded in a viral expression vector.

15. The method of claim 1 or claim 2, wherein the at least one reprogramming factor is an miRNA.

16. The method of claim 1 or claim 2, wherein the somatic cell is a fibroblast.

17. The method of claim 1 or claim 2, wherein the cell is contacted with at least two reprogramming factors.

18. The method of claim 4, wherein the somatic cell is a fibroblast and transdifferentiated to a neuronal cell.

19. The method of any one of claims 1-4, wherein the mechanical force is applied for at least about 24 hours.

20. The method of claim 19, wherein the mechanical force is applied for at least about 15 days.

21. The method of claim 6, wherein the shear force is at least about 1 dyne/cm².

22. The method of claim 21, wherein the shear force is at least about 5 dyne/cm².

23. A system comprising a somatic cell, a media, and a reprogramming factor, wherein the media imposes a shear force on the cell.

24. The system of claim 23, wherein the media imposes the shear force on the cell for at least about 24 hours.

25. The system of claim 23, wherein the media imposes a shear force of at least about 1 dyne/cm².
